(19) United States
(12) Patent Application Publication
(10) Pub. No.: US 2002/0143331 A1
(43) Pub. Date: Oct. 3, 2002

(54) Inter-spinous process implant and method with deformable spacer

(76) Inventors: James F. Zucherman, San Francisco, CA (US); Ken Y. Hsu, San Francisco, CA (US); Charles J. Winslow, Walnut Creek, CA (US); John Flynn, Concord, CA (US)

Correspondence Address: Sheldon R. Meyer, FLIESLER DUBB MEYER & LOVEJOY LLP, Fourth Floor, Four Embarcadero Center, San Francisco, CA, 94111-4156, US

(21) Appl. No.: 10/037,236

(22) Filed: Nov. 9, 2001

Related U.S. Application Data
(63) Continuation-in-part of application No. 09/799,215, filed on Mar. 5, 2001.
(63) Continuation-in-part of application No. 09/473,173, filed on Dec. 28, 1999, now Pat. No. 6,235,030.
(63) Continuation of application No. 09/179,570, filed on Oct. 27, 1998, now Pat. No. 6,048,342.
(63) Continuation-in-part of application No. 09/474,037, filed on Dec. 28, 1999, now Pat. No. 6,190,387.
(63) Continuation of application No. 09/175,645, filed on Oct. 20, 1998, now Pat. No. 6,068,630.
(60) Provisional application No. 60/323,467, filed on Sep. 18, 2001.

(51) Int. Cl. A61B 17/58

(52) U.S. Cl. 606/61

(57) ABSTRACT

The present invention is a interspinous process device which maybe placed between adjacent spinous processes to limit the movement of the vertebrae. The device limits the range of motion of the spinous processes. The various embodiments include spacers which have a compressible medium that preferably provides progressive resistance.

### CLAIM OF PRIORITY

This application claims priority to U.S. Provisional Patent Application entitled INTER-SPINOUS PROCESS IMPLANT AND METHOD WITH DEFORMABLE SPACER, filed Sept. 18, 2001, Ser. No. 60/323,467 and is a continuation-in-part of U.S. patent application Ser. No. 09/799,215 filed on Mar. 5, 2001 ,entitled SPINAL IMPLANTS, INSERTION INSTRUMENTS, AND METHOD OF USE, which is a continuation-in-part of U.S. patent application Ser. No. 09/473,173 filed on Dec. 28, 1999, entitled SPINE DISTRACTION IMPLANT, now U.S. Pat. No. 6,235,030 issued on May 22, 2001, which is a continuation of U.S. patent application Ser. No. 09/179,570 filed on Oct. 27, 1998, entitled SPINE DISTRACTION IMPLANT, now U.S. Pat. No. 6,048,342 issued on Apr. 11, 2000, which is a continuation-in-part of U.S. patent application Ser. No. 09/474,037 filed on Dec. 28, 1999 and entitled SPINE DISTRACTION IMPLANT, now U.S. Pat. No. 6,190,387, issued Feb. 20, 2001, which is a continuation of U.S. patent application Ser. No. 09/175,645 filed on Oct. 20, 1998, entitled SPINE DISTRACTION IMPLANT, now U.S. Pat. No. 6,068,630 issued on May 30, 2000. All of the above are incorporated herein by reference.

### FIELD OF THE INVENTION

The present invention is generally related to an implantable device adapted to distract the spinous process of adjacent vertebrae in order to alleviate the back pain caused by, for example, spinal stenosis and other ailments.

### BACKGROUND OF THE INVENTION

The vertebral column is a bio-mechanical structure composed primarily of ligaments, muscles, vertebrae and intervertebral disks. The bio-mechanical functions of the spine include (1) support of the body, which involves the transfer of the weight and the bending movements of the head, trunk and arms to the pelvis and legs, (2) complex physiological motion between these parts and (3) protection of the spinal cord and the nerve roots.

As the present society ages, it is anticipated that there will be an increase in adverse spinal conditions which are characteristic of older people. By way of example, with aging comes increases in spinal stenosis (including but not limited to central canal and lateral stenosis), the thickening of the bones which make up the spinal column, and the facet arthropathy. Spinal stenosis is characterized by a reduction in the available space for the passage of blood vessels and nerves. Pain associated with such stenosis can be relieved by medication and/or surgery. Of course, it desirable to eliminate the need for major surgery for all individuals and in particular for the elderly.

In addition, there are a variety of other ailments that can cause back pain in patients of all ages. For these ailments it is also desirable to eliminate such pain without major surgery.

Accordingly, there needs to be developed implants for alleviating such conditions which are minimally invasive, can be tolerated by patients of all ages and in particular the elderly, and can be performed preferably on an out patient basis.

### SUMMARY OF THE INVENTION

The present invention is directed to providing a minimally invasive implant for alleviating discomfort associated with the spinal column.

In one aspect of the present invention, the implant reduces and/or eliminates pain by relieving the pressure and restrictions on the aforementioned blood vessels and nerves. Such alleviation of pressure is accomplished by an implant which distracts the spinous processes in order to alleviate the problems caused by spinal stenosis, facet arthropathy and other spinal ailments. While the implant particularly addresses the needs of the elderly, embodiments of the invention can be used with individuals of all ages and sizes where distraction of the spinous processes and/or the maintenance of a spacing between the spinous processes would be beneficial.

Another aspect of the present invention includes an implant with a first support and a second support, having a compressible medium between the first and second support. The compressible medium preferably progressively limits the motion of the adjacent spinous process. The first and second support have a saddle for engaging each spinous process.

Yet another aspect of an embodiment of the present invention is a spacer adapted to be compressed in reaction to forces from a spinous process placed upon the spacer. The spacer has a compressible medium that provides resistance against compression. Such a flexible spacer provides an individual with a larger range of motion.

It is still another aspect of an embodiment of the present invention to include a compressible spacer which prevents wear debris.

Other implants and embodiments within the spirit and scope of the invention can be used to distract the spinous processes, to maintain the distance between the spinous processes and/or to increase the volume of the spinal canal, thereby alleviating restrictions on vessels and nerves associated therewith, and/or pain.

### BRIEF DESCRIPTION OF THE DRAWING

FIGS 1a-1g; FIG. 1a is an assembly view of an embodiment of the invention; FIG. 1b is a side view of the embodiment of the invention of FIG. 1a including a spacer, a main body and a first wing; FIG. 1c is a plane view of the embodiment of the invention in FIG. 1b; FIG. 1d is a side view illustrating the second wing of the embodiment of the invention in FIG. 1a; FIG. 1e is a plane view of the second wing of an embodiment of the invention of FIG. 1a; FIG. 1f is an end view of the spacer of the embodiment of the invention of FIG. 1a; FIG. 1g is a cut-away view illustrating the spacer of the embodiment of the invention of FIG. 1a.

FIG. 2 is aperspective view of still another embodiment of the spacer of the invention;

FIG. 3 is a perspective view of yet another embodiment of the spacer of the invention;

FIG. 4 is a perspective view of still another embodiment of the spacer of the invention;

FIGS. 5a-5b; FIG. 5a is a perspective view of yet another embodiment of the spacer of the invention; FIG. 5b is an end view of the embodiment of the spacer illustrated in FIG. 5a;

FIGS. 6a-6c; FIG. 6a is aperspective view of yet another embodiment of the spacer of the invention; FIG. 6b is a perspective view of the first outer shell of the spacer illustrated in FIG. 6a; FIG. 6c is an end view of the embodiment of the spacer shown in FIG. 6a filled with a deformable or compressible material;

FIG. 7 is a perspective view of yet another embodiment of the spacer of the invention;

FIGS. 8a-8b are perspective views of still other embodiments of the spacer of the invention; and

FIGS. 9a-9b; FIG. 9a is a perspective view of another embodiment of the present invention; FIG. 9b is a cut-away view of the embodiment of the invention illustrated in FIG. 9a.

### DETAIL DESCRIPTION OF THE INVENTION

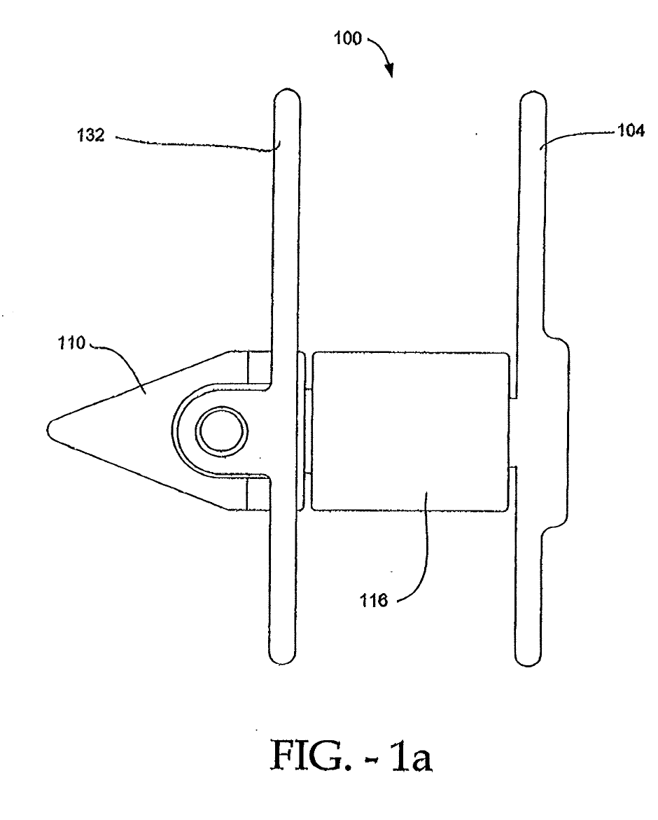

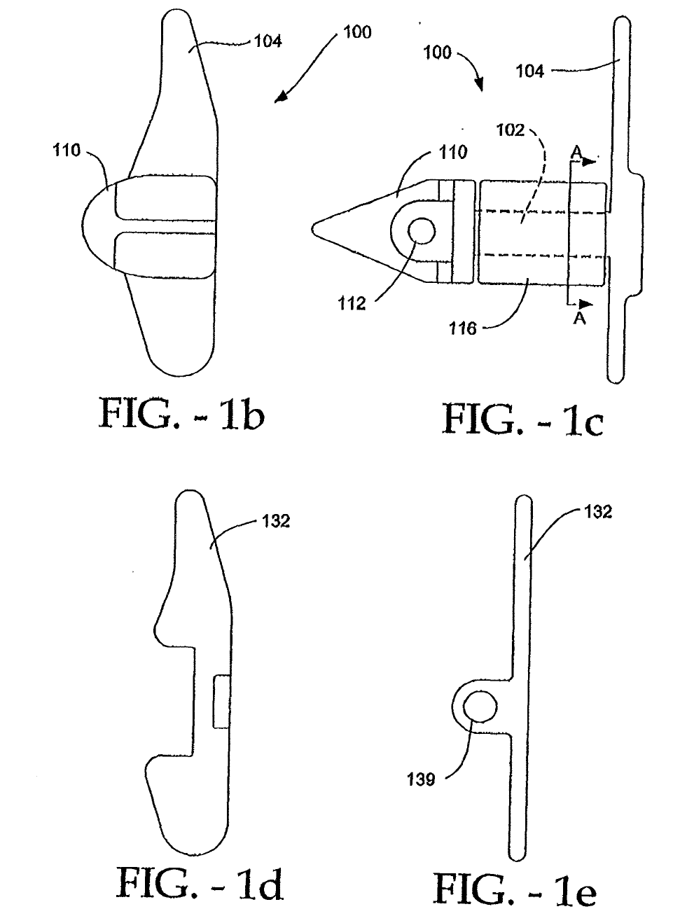

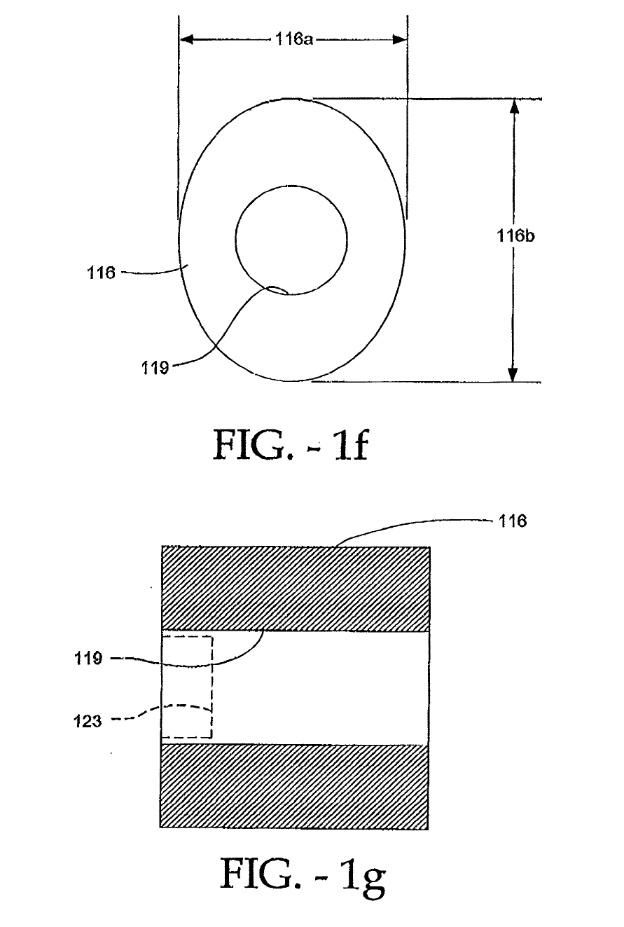

An embodiment of the implant 100 is depicted in FIGS. 1a, lb and 1c. This implant includes the first wing 104 and sleeve 116 and a lead-in and distraction guide 110. This embodiment further includes, as required, a second wing 132 as depicted in FIGS. 1d and 1e. As can be seen in FIG. 1a, a central body 102 extends from the first wing 104. Also, as can be seen in FIGS. 1a and lb, the guide 110 in this particular embodiment is pointed in order to allow the implant to be inserted between, and if necessary distract, adjacent spinous processes.

Additionally, As can be seen in FIGS. 1a, 1f and 1g, the sleeve 116 is preferably cylindrical, and oval or elliptical in shape in cross-section. It is to be understood that sleeve 116 can have other shapes as described throughout the specification and be within the spirit and scope of the invention. Sleeve 116 includes a central bore 119 which extends the length of sleeve 116. The sleeve 116 is received over the central body 102 of the implant 100 and can rotate thereon about the central body 102. In these embodiments, the spacer 116 can preferably have minor and major dimensions as follows:

| Minor Dimension (116a) | Major Dimension (116b) |
|---|---|
| 6 mm | 10 mm |
| 8 mm | 10.75 mm |
| 12 mm | 14 mm |
| 6 mm | 12.5 mm |
| 8 mm | 12.5 mm |
| 10 mm | 12.5 mm |

In another preferred embodiment, the spacer 116 has a cross-section with a major dimension and a minor dimension and the major dimension is greater than the minor dimension and less than about two times the minor dimension.

It is to be understood that the sleeve can be comprised of biologically acceptable material such as titanium or stainless steel. Additionally, it can be comprised of super-elastic material such as an alloy of nickel and titanium. Other structural and material variations for the sleeve are described below.

The advantage of the use of the sleeve 116 as depicted in the embodiment of FIGS. 1a is that the sleeve can be rotated and repositioned with respect to the first wing 104, in the embodiment, in order to more optimally position the implant 100 between spinous processes. It is to be understood that the cortical bone or the outer shell of the spinous processes is stronger at an anterior position adjacent to the vertebral bodies of the vertebra that at a posterior position distally located from the vertebral bodies. Accordingly, there is some advantage of having the implant 100 placed as close to the vertebral bodies as is possible. In order to facilitate this and to accommodate the anatomical form of the bone structures, as the implant is inserted between the vertebral bodies and urged toward the vertebral bodies, the sleeve 116 can be rotated relative to the wings, such as wing 104, so that the sleeve is optimally positioned between the spinous processes, and the wing 104 is optimally positioned relative to the spinous processes. Without this capability, depending on the anatomical form of the bones, it is possible for the wings to become somewhat less than optimally positioned relative to the spinous processes.

As required, the implant 100 can also include a second wing 132 which fits over the guide 110 and is preferably secured by a bolt through apparatus 134 of second wing 132 to the threaded bore 112 located in guide 110. As implanted, the first wing 104 is located next to the adjacent first side of the spinous processes and the second wing 132 is located adjacent to second side of the same spinous processes.

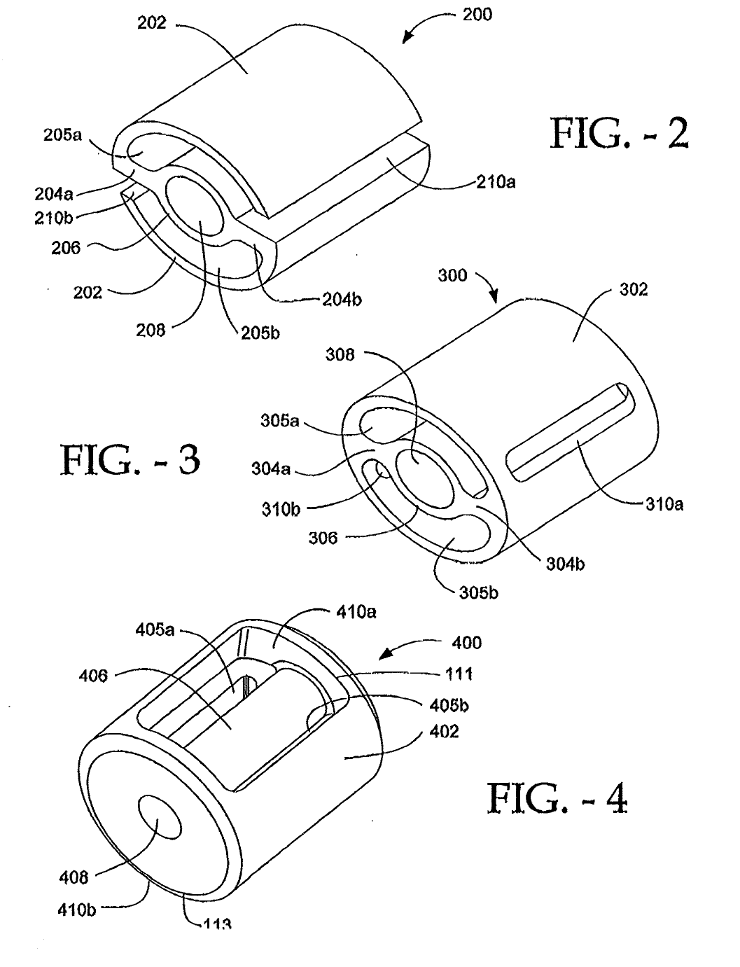

Referring now to FIGS. 2-8, various embodiments of spacers adapted for placing between the first wing 104 and the second wing 132 are shown. The preferred material for the various spacers described below is titanium in combination with a deformable material such as silicone. It is within the scope of the present invention to manufacture the spacers from other biologically acceptable material such as, by way of example only, stainless steel or an alloy of nickle and titanium along with another deformable material such as another deformable polymer.

Turning now to FIG. 2, the spacer 200 includes an outer shell 202. The outer shell 202 is integrally formed with the center shaft 206 by two support columns 204. The center shaft has a bore 208 extending through. Each support column 204 extends substantially perpendicular from the center shaft 206. Between the outer shell 202 and the center shaft 206, a cavity 205 is created.

The shape of the outer shell 202 as shown in FIG. 2 is elliptical in shape. It is within the scope of the invention that the outer shell 202 may comprise other shapes such as, but not limited to, a cylindrical or egg shape. Regardless of the shape, the outer shell 202 is not continuous in this preferred embodiment. One half of the outer shell 202 extends from the end of one support column 204a and around the center shaft 206 until the outer shell 202 almost reaches the second support column 204b. The second half of the outer shell 202 is the same as the first half, and in this case both halves extend in a clockwise direction. Since each half of the outer shell 202 extends from a different support column 204, two slots 210a and 210b are created. Both slots 210a,b extend along the length of the spacer 200. The slots 210 function to lower the rigidity of the outer shell 202 so that the outer shell 202 is more flexible and functions as a cantilever spring. The smallest diameter of the space (circular or elliptical) can preferably range from 6 mm. to 11 mm. The thickness of the outer shell can preferably be 2 mm. The spacer can have other dimensions as identified previously.

Preferably, a compressible substance 207 is placed into the cavities 205a,b located between the outer shell 202 and the center shaft 206. The compressible substance 207 provides resistance against the outer shell 202 traveling towards the center shaft 206. As previously mentioned, the compressible substance in this embodiment is preferably silicone. It is within the scope of the invention that the compressible substance 207 may comprise another medium such as, but not limited to, urethane-coated silicone and/or co-formed with silicone so that the urethane will not be attacked by the body, or another ultra-high molecular weight polymer. Another preferred material is polycarbonate-urethane, a thermoplastic elastomer formed as the reaction product of a hydroxl terminated polycarbonate, an aromatic diisocyanate, and a low molecular weightglycol used as a chain extender. A preferred polycarbonate glycol intermediate, poly (1,6-hexyl 1,2-ethyl carbonate) diol, PHECD, is the condensation product of 1,6-hexanediol with cyclic ethylene carbonate. The polycarbonate macroglycol is reacted with aromatic isocyanate, 4,4′-methylene bisphenyl diisocyanate (MDI), and chain material is preferable used at a hardness of 55 durometer. This material, as well as the other materials, can be used in the other embodiments of the invention.

The compressible medium preferably has a graduated stiffness to help gradually distribute the load when a spinous processes places a force upon the outer shell 202. For example, the hardness of the silicone can be the lowest where the silicone contacts the outer shell 202, and the hardness of the silicone can be the highest where the silicone contacts the center shaft 206. Alternatively, the silicone can have a higher hardness in the center of the silicone located between the outer shell 202 and the center shaft 206.

The compressible medium 207 fills the cavity between the outer shell 202 and the center shaft 206 and is flush with the outer shell 202. When the spacer 200 is inserted between adjacent spinous processes, the outer shell 202 protects the compressible substance (e.g., silicone) from directly contacting the spinous processes because the slots 210 are along the side of the spacer 200. Therefore, the deformable material 207 does contact the spinous processes and wear debris is reduced or eliminated.

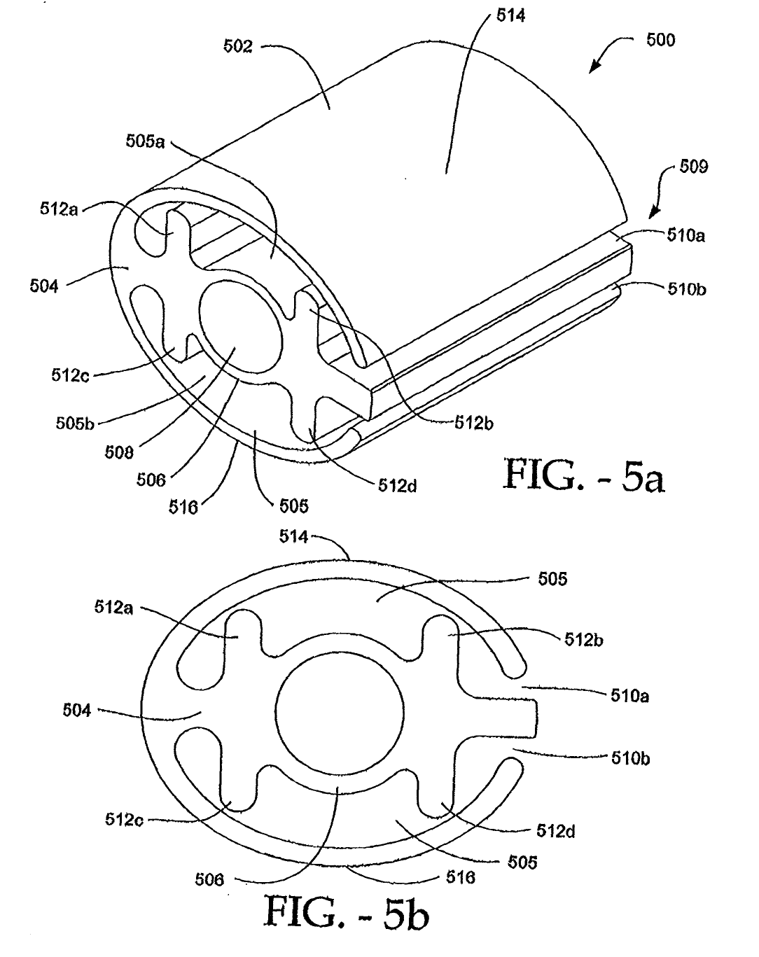

It is to be understood that for this and also in the embodiments in FIGS. 3, 5a and 5b, the embodiment can be constructed without a compressible material, with the outer shell solely providing the flexibility of the spacer. It is also to be understood that the embodiments shown in FIGS. 3-8 can have the dimensions and be made of the materials similar to those of FIG. 2. It is additionally to be understood that the metal components of any of the embodiments hereof can be comprised of a suitable plastic or composite material including fibers for strength.

Now referring to FIG. 3, the spacer 300 has an outer shell 302 and a center shaft 306. The center shaft 306 has a bore 308 extending through. The center shaft 306 is connected with the outer shell 302 by two support columns 304a,b, with each support column 304a,b located on opposite sides of the center shaft 306. Similar to the embodiment of the present invention as illustrated in FIG. 2, the outer shell 302 is elliptical, yet may comprise other shapes such as , but not limited to, a cylindrical or egg shape.

The outer shell 302 has two slots 310a,b. The slots 310a,b extend through the wall of the outer shell 302 to form a rectangular-like opening. It is within the scope of the invention for the spacer 300 to have more than two slots 310 and with different shapes. The slots 310a,b are used to make the outer shell 302 more flexible. It is preferred that the slots 310a,b are located on the sides of the spacer 300 so that none of the slots 310a,b contact a spinous process.

Between the outer shell 302 and the center shaft 306 are two cavities 305a,b. These cavities are separated by the support columns 304a,b. The two cavities created between the outer shell 302 and the center shaft 306 preferably have a compressible substance therein. As previously mentioned, the compressible substance is preferably silicone. To improve the load distribution upon the outer shell 302 and ease the load on the spinous processes, the silicone can have a graduated stiffness. For example, the hardness of the silicone can be the lowest where the outer shell 302 contacts the silicone, and the hardness of the silicone can be the highest where the center shaft 306 and the support column 304 contacts the silicone. Alternatively, the silicone can have a higher hardness in the center of the silicone riding between the outer shell 302 and the center shaft 306.

The silicone is placed between the outer shell 302 and the center shaft 306 so that the silicone extends into the slots 310 and is flush with the outer shell 302. Since the spinous processes do not directly contact the silicone, this embodiment of the present invention also helps prevent wear debris.

Referring now to FIG. 4, yet another embodiment of the present invention includes spacer 400. The spacer 400 has an outer shell 402 and a center shaft 406. The center shaft 406 has a bore 408 extending through. The spacer 400 has two openings 410a,b that are substantially along the top 111 and bottom 113 portions of the outer shell 402. Between the outer shell 402 and the center shaft 406, cavities 405a,b are created which connects the two openings 410a,b.

Similar to the previous embodiments, a compressible medium such as silicone is placed into the cavity 405a,b and openings 410a,b until the silicone becomes flush with the outer shell 402. Preferably, the silicone also has a graduated stiffness. For example, the hardness of the silicone can be the lowest where it is flush with the outer shell 402, and can be the highest where the silicone contacts the center shaft 406. Unlike the previous embodiments, the exposed silicone will directly contact the spinous processes.

Referring now to FIGS. 5a-5b, another embodiment of the invention is spacer 500. The spacer 500 has an outer shell 502 and a center shaft 506. The outer shell 502 forms a “C”-like shape. The center shaft 506 has a bore 508 extending through. The center shaft 506 is attached to the outer shell 502 by a support 504. The support 504 is substantially horizontal extending from the vertical center of the “C” to the middle of the open end 509. The outer shell 502 defines two slots 510a,b along the length of the open end 509. Both slots 510a,b are defined by the space between the support 504 and each end portion of the outer shell 502. Since the outer shell 502 is fixed at one end only, the outer shell 502 functions like a cantilever-type spring. The outer shell 502 is shown as elliptical in shape. It is within the scope of the present invention that the spacer 500 may comprise other shapes such as, but not limited to, a cylindrical or egg shape.

The support 504 has preferably at least two protrusions such as protrusions selected from protrusions 512a,b,c,d. For example, the spacer 500 in FIGS. 5a,b has four protrusions 512a,b,c,d. Each protrusion 512a,b,c,d extends substantially and preferably perpendicular in this embodiment from the support 504 towards the inner surface of the outer shell 502. While the spacer 500 is in anon-compressed state, there is a gap between each protrusion 512a,b,c,d and the outer shell 502. When the spacer 500 is compressed, the protrusions 512a,b,c,d function to restrict the deflection of the outer shell 502. When a spinous process exerts a force upon the outer shell 502, the outer shell 502 will deflect toward the center shaft 506 until the outer shell 502 contacts the protrusion 512a,b,c,d. Essentially, the protrusions 512a,b,c,d, function as a stop mechanism preventing the outer shell 502 from deflecting too much, and thus limiting the motion of the spinous processes.

Similar to the previous embodiments, cavities 505a,b are formed between the center shaft 506 and the outer shell 502. A compressible substance such as silicone is placed within the cavity 505. It is preferable that the silicone have a graduated stiffness to help distribute the load placed upon the outer shell 502. For example, the hardness of the silicone can be the lowest where the silicone contacts the inner surface of the outer shell 502, and the hardness of the silicone can be the highest where the silicone contacts the center support shaft 506, and the support 504 and the protrusions 512a,b,c,d. Alternatively, the silicone can have a higher hardness in the center of the silicone rising between the outer shell 502 and the center shaft 506.

The silicone fills the cavities 505a,b until the silicone is flush with the outer shell 502. When the spacer 500 is inserted between adjacent spinous processes, the top and bottom portions 514, 516 of the spacer 500 contact the spinous process. Therefore, the silicone will not directly contact the spinous processes which aids in the prevention of wear debris.

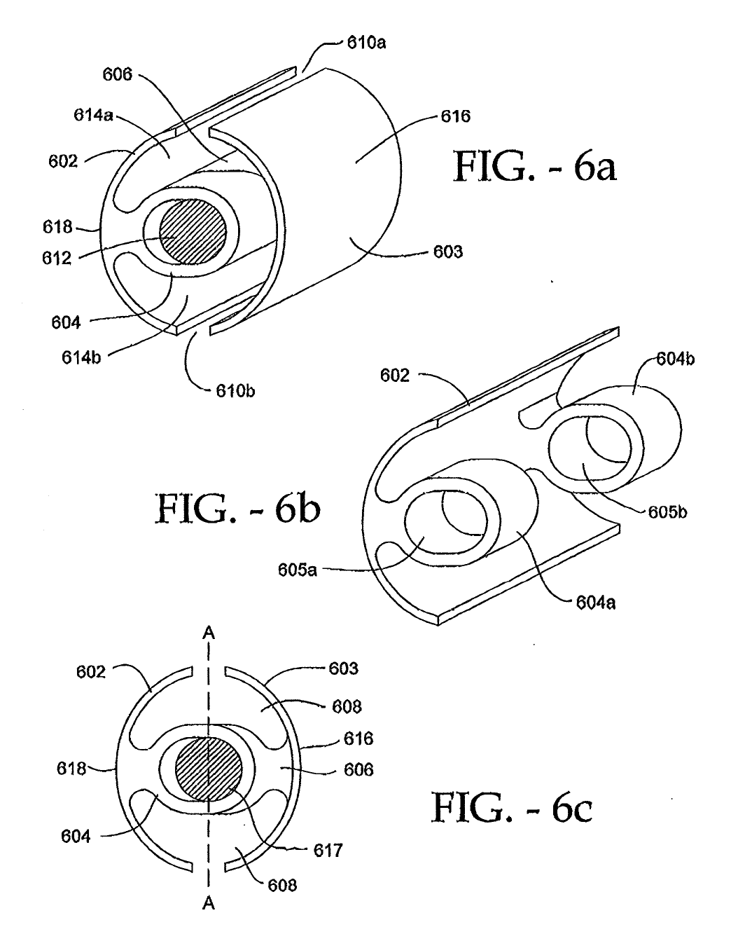

Referring now to FIGS. 6a-6c, another embodiment of the present invention is spacer 600. The spacer 600 has a first outer shell 602 and a second outer shell 603. The first outer shell 602 has at least two support elements 604a,b. Each support element 604a,b has a bore 605a,b extending therethrough. The support elements 604a,b are located substantially at either end of the first outer shell 602 along a single horizontal axis. The bores 605a,b are oval in a preferred embodiment. This shape allows the spacer 600 to move relative to the central shaft or axis (FIG. 1) upon which the spacer is mounted. The second outer shell 603 has a single support element 606, located substantially in the center of the second outer shell 603 and along the same horizontal axis as the two support elements 604a,b. The support element 606 also has a bore extending through which is similar to bore 605. Support element 606 is located between support element 604a,b in FIG. 6a. A central shaft 612 (shaft 102 in FIG. 1c) is placed through the support elements 604a,b, 606 to form a hinge-type connection between the first outer shell 602 and the second outer shell 603 (see FIG. 6a). The hinge-type connection allows the first outer shell 602 and the second outer shell 603 to move independently of each other.

When the first outer shell 602 and the second outer shell 603 are connected by shaft 612, slots 610a,b are created along the side edges of the spacer 600. Two cavities 614a,b are also created, defined by the hinge-type connection between the first outer shell 602 and the second outer shell 603. Similar to the previous embodiments, a compressible substance (e.g., silicone) can fill each cavity and extend into the slots 610a,b until the silicone is flush with the first outer shell 602 and the second outer shell 603. Additionally, it is preferred that the silicone have a graduate hardness similar to the previous embodiments. In one embodiment, the hardness of the silicone can be the highest along view line A-A, and can be the lowest where the silicone contacts the first and second outer shell 602, 603. Alternatively, the silicone can have the highest hardness where it contacts the support elements 604a,b, 606, and can have the lowest hardness where the silicone fills the slots 610a,b.

When the spacer 600 is inserted between adjacent spinous process, only the top and bottom portions 616, 618 of the spacer 600 will directly contact each spinous process. Therefore, the first outer shell 602 and the second outer shell 603 prevent direct contact between the silicone and the spinous process. Accordingly, the spacer 600 helps prevent wear debris from being formed.

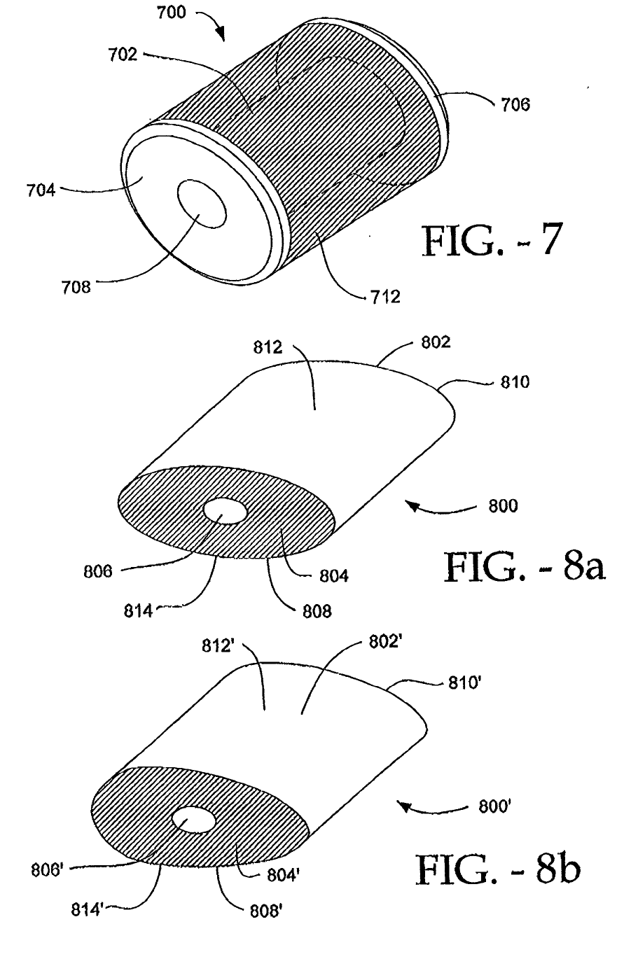

Now referring to FIG. 7, still yet another embodiment of the present invention is spacer 700. Spacer 700 includes preferably a component in the shape of an elliptical or oval or cylindrical spool 710. Alternatively, the component 700 can be formed for method or suitable plastic material or composites including, by way of example only, fibers for strength. The spacer 700 has a center shaft 702 with a bore 708 extending through. As in other embodiments the bore 708 can be, by way of example only, circular, oval or elliptical. A first end 704 and a second end 706 are integrally formed with the center shaft 702 in this preferred embodiment. Both the first end 704 and the second end 706 extend outward from the center shaft 702 and form a circular rim around each end of the center shaft 702. It is within the scope of the present invention for the first end 704 and second end 706 to comprise other shapes such as, but not limited to, elliptical, circular, oval or egg-shaped.

A compressible medium 712 surrounds the center shaft 702. As previously mentioned, the compressible substance is preferably silicone. The silicone extends out from the center shaft 702 until it is flush with the outer rim of both the first end 704 and the second end 706. With the silicone around the center shaft 702, the spacer 700 will look like an elliptical cylinder in this embodiment. The spacer 700 does not have an outer shell surrounding the silicone. When the spacer 700 is inserted between adjacent spinous process, the silicone will directly contact the spinous process. A preferred embodiment of the spacer 700 will have silicone with a graduated stiffness to help distribute the load placed upon the spacer 700. For example, the hardness of the silicone can be the lowest at the outermost surface that contacts the spinous process, and the hardness of the silicone can be the highest where the silicone surrounds and contacts the center shaft 702. Alternatively the hardness can be greater where the silicone contacts the spinous process and then less hard adjacent to the center shaft 702.

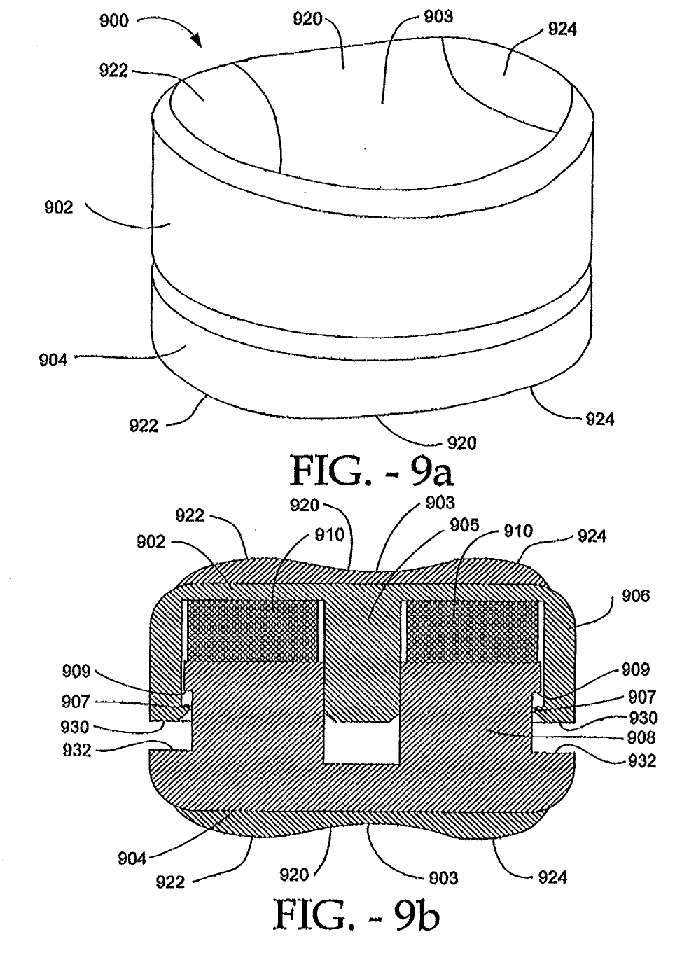

Now turning to FIG. 8a, another embodiment of the present invention is spacer 800. The spacer 800 has an outer shell 802 which can be metallic or plastic. The outer shell 802 is preferably elliptical in shape. It is within the scope of the present invention that the outer shell 802 can be a shape such as, but not limited to, a cylindrical or egg shape. Regardless of the shape, the outer shell 802 is open on both ends 808, 810.

A compressible substance 804 is placed within the outer shell 802 and is flush with both ends 808, 810 of the outer shell 802. A bore 806 extends through the compressible substance 804. If desired the bore can be defined by a metallic or plastic sleeve held on the compressible substance 804. Similar to the previous embodiments, the compressible substance 804 is preferably silicone. A preferred embodiment of the spacer 800 has silicone with a graduated stiffness. In an embodiment, the hardness of the silicone can be the highest at the bore 806, and the hardness of the silicone can be the lowest where the silicone contacts the inner surface of the outer shell 802. Alternatively, the hardness of the silicone can be the highest adjacent shell and lowest about bore 806.

When the spacer 800 is inserted between adjacent spinous processes, only the top and bottom portions 812, 814 will directly contact each spinous process. Therefore, the outer shell 802 prevents direct contact between the silicone and the spinous processes. Accordingly, the spacer 800 helps prevent wear debris from being formed.

By way of example only, the thickness of the outer shell can be about 0.010 inches with the hardness of the compressible medium being about 50 durometer. By way of example only, the outer shell can be about 0.010 inches with the hardness of the compressible medium being about 70 durometer.

It is also to be understood that the spacer 800 can include any of the compressible medium 804 discussed herein and/or compatible with the body, with a bore hole provided therethrough. That is to say that the outer shell 802 can be eliminated in this embodiment. Preferably the spacer is comprised of silicone, however, other materials are within the spirit and scope of the invention. FIG. 8b depicts an egg-shaped spacer 800′ with a bore 809′. The spacer 800′ is comprised of a compressible medium.

Referring now to FIGS. 9a-9b, the interspinous process device on implant 900 has a first support 902 and a second support 904. The first support 902 and the second support 904 directly contact the spinous process and can be made of a suitable metal or a suitable plastic. Both the first support 902 and the second support 904 have a contour 903. The contour 903 allows the device 900 to be contoured to and to engage each spinous process so, preferably, that the device 900 does not move laterally. Each contour 903 includes a concave portion 920 and upstanding ridges 922, 924. It is to be understood that the ridges can be higher than shown in FIG. 9a in order to define a deeper contour. Additionally, ridges 924, especially when higher, of supports 903, 904 together can define a first wing and ridges 922, especially when higher, of support 903, 904 define a second wing. Such wings can function in much the same way as the wings described in other embodiments of the invention.

During the method of implanting device 900, both spinous processes are exposed using appropriate surgical techniques, and thereafter the device 900 is positioned so that the saddles 903 of both the first support 902 and the second support 904 engage the respective spinous process. The concave shape of the saddle 903 distributes the forces between the saddle 903 and the respective spinous process. This ensures that the bone is not reabsorbed due to the placement of the device 900 and that the structural integrity of the bone is maintained.

Referring now to FIG. 9b, the first support 902 has a female receiving mechanism 906 and the second support 904 has a male engaging mechanism 908. The width of the female receiving mechanism 906 and the male engaging mechanism 908 are substantially similar. The female receiving mechanism 906 further has an alignment column 905 to assist in the movement of the supports 902, 904 relative to each other.

The first support 902 and the second support 904 are interlocked so that the first support 902 and the second support 904 cannot be independently separated. The first support 902 has a ledge 907 that extends around the inner circumference of the first support 902. Similarly, the second support 904 has a ledge 909 extending around the circumference of the male engaging mechanism 908. If the first support 902 and the second support 904 travel in opposite directions, the ledges 907 and 909 will eventually engage and prevent the first support 902 and the second support 904 from separating. Preventing the first support 902 and the second support 904 from separating also contains the compressible medium 910, as described below, within the device 900.

Placed within the female receiving mechanism 906 is a compressible medium 910. As previously mentioned the compressible medium 910 provides resistance, limiting the possible range of motion of the spinous process. By way of example only, the compressible medium 910 can be silicone. It is within the scope of the present invention that the compressible medium can include, by way of example only, a spring mechanism, a synthetic gel or a hydrogel. The compressible or deformable material can also include material which offers, for example, increased resistance to compression the more the material is compressed. For example, as compression and deformation occur, the material can offer a steady resistive force or a resistance force that increases, either linearly or non-linearly, the more the implant is compressed.

With respect to an embodiment with a graduated stiffness, the hardness of the silicone can be the lowest where the first support 902 contacts the silicone, and the hardness of the silicone 910 can be the highest where the second support 904 contacts the silicone. Alternatively, the silicone can have a higher hardness in the center of the silicone riding between the supports 902, 904.

In this and with the other embodiments, the medium 910 can also be designed to vary resistance to movement according to the speed or rate of deformation. For example, when an individual leans back slowly, the adjacent spinous processes place a force onto the first support 902 and the second support 904. With slow backward bending the force is small and gradual until the limit of compression of the material is reached. However, if the individual attempts a rapid activity that can result in a severe first compression of the device 900, the medium 910 can offer higher stiffness, preventing the spinous processes from making excessive motion and causing pain.

Preferably, the height of the device 900 is slightly larger than the undistracted distance between the adjacent spinous processes. When the device 900 is then inserted between the spinous process, the contours 903 will press against each spinous process and assist to keep the device 900 in place. During a daily routine, an individual will perform functions that will translate into vertical movement of each spinous process. It is important that the individual be able to retain some of his normal range of motion. To retain a normal range of motion, the device 900 can preferably be compressed when the spinous processes place a force upon the first support 902 and the second support 904. Thus, when the device 900 is in a normal state the outer peripheral edge 930, 932 of first and second support 902, 904 respectively do not contact each other. However, ridges 930, 932 act as a limit to the amount device 900 can be compressed. Such an arrangement reduces potential resorption of the bone adjacent to the implant and to more gradually limit extension or backward bending of the spinal column.

The embodiment of this implant as well as the several other implants described herein act to limit extension. These implants, however, do not inhibit the flexion of the spinal column when the spinal column is bent forward.

The foregoing description of preferred embodiments of the present invention has been provided for the purposes of illustration and description. It is not intended to be exhaustive or to limit the invention to the precise forms disclosed. Many modifications and variations will be apparent to the practitioner skilled in the art. The embodiments were chosen and described in order to best explain the principles of the invention and its practical application, thereby enabling others skilled in the art to understand the invention and the various embodiments and with various modifications that are suited to the particular use contemplated. It is intended that the scope of the invention be defined by the following claims and their equivalence.

1. An implant adapted for placing between spinous processes, the implant comprising:
a body with a shaft extending therefrom;
a spacer that is rotatably mounted on said shaft;
said spacer including:
(a) a spacer shaft having a bore extending through;
(b) an outer shell with a cavity between the spacer shaft and the outer shell; and
(c) a compressible medium inserted into the cavity between the spacer shaft and the outer shell.

2. The implant as recited in claim 1, wherein the outer shell is connected with the spacer shaft by at least a support column extending from the spacer shaft.

3. The implant as recited in claim 1, wherein the outer shell is elliptical in shape.

4. The implant as recited in claim 1, wherein the outer shell is cylindrical in shape.

5. The implant as recited in claim 1, wherein the outer shell is egg-shaped.

6. The implant as recited in claim 1, wherein the outer shell has at least one slot, said slot extending at least part way along the length of the outer shell to weaken the rigidity of the outer surface.

7. The implant as recited in claim 6, wherein the compressible medium fills in the slot and is substantially flush with the outer edge of the outer shell.

8. The implant as recited in claim 1, wherein the compressible medium inserted into the cavity between the spacer shaft and the outer shell is silicone.

9. The implant as recited in claim 1, wherein the compressible medium is a high molecular weight polymer.

10. The implant as recited in claim 1, wherein the compressible medium has a graduated stiffness.

11. The implant as recited in claim 1, wherein the outer shell protects the compressible medium from directly contacting the spinous processes.

12. The implant as recited in claim 1, wherein the outer shell has at least two openings extending along at least part of the length of the outer shell; and said openings communicate with the cavity and are filled with the compressible medium, with the compressible medium in the openings adapted to contact the spinous processes.

13. The implant as recited in claim 12, wherein the compressible medium has a graduated stiffness.

14. An implant adapted for placing between spinous processes, the implant comprising:
a body with a shaft extending therefrom;
a spacer that is rotatably mounted on mid shaft;
said spacer formed in the shape of a spool and including:
(a) a spacer shaft, having a bore extending through;
(b) a first end having an outer edge and extending from the spacer shaft;
(c) a second end having an outer edge and extending from the spacer shaft; and
(d) a compressible medium surrounding the spacer shaft.

15. The implant as recited in claim 14, wherein the first and second ends are circular in shape.

16. The implant as recited in claim 14, wherein the first and second ends are elliptical in shape.

17. The implant as recited in claim 14, wherein the compressible medium is silicone.

18. The implant as recited in claim 14, wherein the compressible medium is adapted to contact the spinous process when the spacer is inserted between adjacent spinous processes.

19. The implant as recited in claim 14, wherein the compressible medium has a graduated stiffness.

20. An implant adapted for placing between spinous processes, the spacer comprising:
a body with a shaft extending therefrom;
a spacer that is rotatably mounted at said shaft;
said spacer including:
(a) a first outer shell having at least one support element, said support element having a bore extending therethrough;
(a) a second outer shell having at least one support element, with the support element of the first outer shell and the support element of the second outer shell, and with the bores of the first and second support elements having the shaft extending through, forming a hinge-type connection;
(c) a cavity located between the first outer shell and the second outer shell; and
(d) a compressible medium located into the cavity between the first and second outer shell.

21. The implant as recited in claim 20, wherein the compressible medium is silicone.

22. The implant as recited in claim 20, wherein the bore extending through each support element is one of elliptical, oval and circular in shape.

23. The implant as recited in claim 20, wherein the first and second outer shell create two slots extending at least partially along the length of the spacer.

24. The implant as recited in claim 20, wherein the stiffness of the compressible medium is graduated.

25. The implant as recited in claim 20, wherein the first and second outer shells are adapted to protect the compressible medium from contacting the spinous processes.

26. An implant adapted for placing between spinous processes, the implant comprising:
a body with a shaft extending therefrom;
a spacer that is rotatably mounted at said shaft;
said spacer including:
(a) an outer shell creating a cavity; and
(b) a compressible medium filling the cavity, having a bore extending through the compressible medium, with the shaft received through the bore.

27. The implant as recited in claim 26, wherein the outer shell is elliptical in shape.

28. The implant as recited in claim 26, wherein the outer shell is cylindrical in shape.

29. The implant as recited in claim 26, wherein the compressible medium is silicone.

30. The implant as recited in claim 26, wherein the compressible medium is a high molecular weight polymer.

31. The implant as recited in claim 26, wherein when the thickness of the outer shell is 0.020′, the hardness of the compressible medium is approximately 50 durometer.

31. The implant as recited in claim 26, wherein when the thickness of the outer shell is 0.010′, the hardness of the compressible medium is approximately 70 durometer.

32. The implant as recited in claim 26, wherein the hardness of the compressible medium is graduated, and wherein the hardness of the compressible medium is the lowest where the compressible medium contacts the outer shell, and the hardness of the compressible medium is the highest where the compressible medium is located adjacent to the bore.

33. An implant adapted for placing between spinous processes, the spacer comprising:
a body with a shaft extending therefrom;
a spacer rotatably mounted on said shaft;
said spacer having:
(a) an outer shell;
(b) a support communicating with the outer shell and a cavity formed between the outer shell and the support , with a bore extending through the support, said shaft received in said bore;
(c) at least one protrusion extending from the support to restrict a deflection of the outer shell toward said support; and
(d) a compressible medium within the cavity formed between the outer shell and the support.

34. The implant as recited in claim 33, wherein the compressible medium is silicone.

35. The implant as recited in claim 33, wherein the compressible medium is a high molecular weight polymer.

36. The implant as recited in claim 33, wherein the outer shell is elliptical in shape.

37. The implant as recited in claim 33, wherein the outer shell is oval in shape.

38. The implant as recited in claim 33, wherein the outer shell is egg-shaped.

39. The implant as recited in claim 33, wherein the outer shell is cylindrical in shape.

40. The implant as recited in claim 33, wherein the hardness of the compressible medium is graduated, wherein the hardness of the compressible medium is the lowest where the compressible medium contacts the outer shell, and the hardness of the compressible medium is the highest where the compressible medium contacts the center support.

41. The implant as recited in claim 33, wherein the spacer further has two slots extending along the length of the outer shell.

42. An implant adapted for placing between adjacent spinous processes, the implant comprising:
a body with a shaft extending therefrom;
a spacer that is rotatably mounted on said shaft;
said spacer having:
(a) a support, having a bore extending through;
(b) an outer shell connected with the support, creating a cavity between the support and the outer shell; and
(c) a compressible medium within the cavity between the support and the outer shell, where the hardness of the compressible substance is graduated.

43. The implant as recited in claim 42, wherein the compressible substance is silicone.

44. The implant as recited in claim 42, wherein the outer shell is elliptical in shape.

45. The implant as recited in claim 42, wherein the outer shell is cylindrical in shape.

46. The implant as recited in claim 42, wherein the outer shell has at least one slot.

47. The implant as recited in claim 42, wherein the compressible medium extends into the slot so that the compressible medium is flush with the outer shell.

48. The implant of claim 42, wherein, the hardness of the compressible substance is the lowest where the compressible substance contacts the outer shell, and the hardness of the compressible substance is the highest where the compressible substance contacts the support.

49. An implant for placing between spinous processes, the implant comprising:
a body with a shaft extending therefrom;
a spacer that is rotatably mounted on said shaft;
said spacer including:
(a) a spacer shaft having a bore extending therethrough;
(b) an outer shell located about the spacer;
(c) first and second openings located in the outer shell:
(d) a compressible medium located in the first and second openings, with the compressible medium adapted to contact the spinous processes with the implant placed between the spinous processes.

50. The implant of claim 49 wherein said outer shell is one of elliptical, oval, cylindrical and egg-shaped.

51. The implant of claim 49 wherein said compressible medium has a graduated stiffness.

52. The implant of claim 49 wherein said compressible medium is silicone.

53. The implant of claim 49 wherein said compressible medium is a high molecular weight polymer.

54. An implant for placing between spinous processes, the implant comprising:
a body with a shaft extending therefrom;
a spacer that is rotatably mounted on said shaft; and
said spacer including:
(a) a first outer shell with first and second supports extending therefrom, with each said first and second supports including a bore defined therein;
(b) a second outer shell with a third support extending therefrom, with the third support including a bore defined therein;
(c) the third support located between the first and second support with the bores, with the shaft received through the bores of the first, second, and third supports;
(d) a cavity located between the first and second outer shells; and
(e) a compressible medium located in the cavity.

55. The implant as recited in claim 54, wherein the compressible medium is silicone.

56. The implant as recited in claim 54, wherein the bore extending through each support element is one of elliptical, oval and circular in shape.

57. The implant as recited in claim 54, wherein the first and second outer shell create two slots extending at least partially along the length of the spacer.

58. The implant as recited in claim 54, wherein the stiffness of the compressible medium is graduated.

59. The implant as recited in claim 54, wherein the first and second outer shells are adapted to protect the compressible medium from contacting the spinous processes.

60. An implant for placing between spinous processes, the implant comprising:
a body with a shaft extending therefrom;
a spacer that is rotatably mounted on said shaft; and
said spacer including a compressible medium with a bore provided therethrough, with the shaft received in said bore, such that the spacer can rotate relative to said shaft.

61. The implant of claim 60 wherein said spacer is cylindrical in shape.

62. The implant of claim 60 wherein said spacer is elliptical in shape.

63. The implant of claim 60 wherein said spacer is oval in shape.

64. The implant of claim 60 wherein said space is egg-shaped.

65. The implant of claim 60 wherein said compressible medium is silicone.

66. The implant of claim 60 wherein said compressible medium is a high molecular weight polymer.

67. The implant of claim 60 wherein the hardness of the compressible medium is graduated from less hard at a distance from the bore to more hard closer to the bore.

68. An implant adapted for placing between spinous processes, the implant comprising:
a body with a shaft extending therefrom;
a spacer rotatably mounted on the shaft; and
said spacer having:
(a) an outer shell;
(b) a support communicating with the outer shell and a cavity formed between the support and the outer shell, with a bore formed in the support and with the shaft received in said bore; and
(c) at lease one protrusion extending from the support to restrict a deflection of the outer shell toward the support.

69. The implant as recited in claim 68, wherein the spacer contains a compressible medium.

70. The implant as recited in claim 68, wherein the outer shell is elliptical in shape.

71. The implant as recited in claim 68, wherein the outer shell is oval in shape.

72. The implant as recited in claim 68, wherein the outer shell is egg-shaped.

73. The implant as recited in claim 68, wherein the hardness of the compressible medium is graduated, the hardness of the compressible medium is the lowest where the compressible medium contacts the outer shell, and the hardness of the compressible medium is the highest where the compressible medium contacts the center support.

74. An implant adapted for placing between spinous processes, the implant comprising:
a body with a shaft extending therefrom;
a spacer rotatably mounted on the shaft; and
said spacer having:
a support having a bore provided therethrough, with the shaft received in the bore;
an outer shell cantilevered from the support, the outer shell adapted to contact a spinous process.

75. The implant of claim 74 including:
a second outer shell cantilevered to said support.

76. The implant of claim 74 including:
a compressible medium located between the support and the outer shell.

77. The implant of claim 74 including:
a protrusion extending from the support toward the outer shell in order to limit a deflection of the outer shell toward the support.

78. The implant of claim 74 wherein said support includes a first portion and a second portion and wherein said outer shell is cantilevered from said first portion and another outer shell is cantilevered from said second portion.

79. The implant of claim 75 wherein said first and second outer shells are cantilevered from the support from the same location.

80. The implant of claim 75 wherein said first and second outer shells are cantilevered from the support form the same location, but extend in opposite directions.

81. The implant of claim 75 wherein said first and second outer shells together form the shape of one of an ellipse, an oval, a circle, and an egg.

82. The implant of claim 78 wherein said first and second outer shells extend circumferentially about the support.

83. The implant of claim 78 wherein said first and second outer shells extend one of clockwise and counterclockwise about the support.

84. The implant of claim 80 wherein the first and second outer shells are located about and spaced from the support, and wherein the first and second outer shells extend toward each other from the same location on the support.

85. An implant adapted to be positioned between spinous processes, the implant including:
a first support defining a first saddle adapted to receive a spinous process;
a second support defining a second saddle adapted to receive another spinous process;
the first and second supports mated together to form an enclosed cavity;
a compressible medium located in the cavity.

86. The implant of claim 85 wherein said compressible medium includes silicone.

87. The implant of claim 85 wherein said compressible medium includes a high molecular weight polymer.

88. The implant of claim 85 wherein said first and second supports are mated together so as to limit movement of the first and second supports both toward each other and away from each other.

89. The implant of claim 85 wherein said first support has a first peripheral edge and the second support has a second peripheral edge with the first peripheral edge associated with the second peripheral edge so to limit movement of the first and second supports both toward each other and away from each other.

90. The implant of claim 1 wherein the compressible medium is a thermoplastic elastomer.

91. The implant of claim 14 wherein the compressible medium is a thermoplastic elastomer.

92. The implant of claim 20 wherein the compressible medium is a thermoplastic elastomer.

93. The implant of claim 26 wherein the compressible medium is a thermoplastic elastomer.

94. The implant of claim 33 wherein the compressible medium is a thermoplastic elastomer.

95. The implant of claim 43 wherein the compressible medium is a thermoplastic elastomer.

96. The implant of claim 49 wherein the compressible medium is a thermoplastic elastomer.

97. The implant of claim 60 wherein the compressible medium is a thermoplastic elastomer.

98. The implant of claim 85 wherein the compressible medium is a thermoplastic elastomer.

99. The implant of claim 1 wherein the compressible medium is polycarbonate urethane.

100. The implant of claim 14 wherein the compressible medium is polycarbonate urethane.

101. The implant of claim 20 wherein the compressible medium is polycarbonate urethane.

102. The implant of claim 26 wherein the compressible medium is polycarbonate urethane.

103. The implant of claim 33 wherein the compressible medium is polycarbonate urethane.

104. The implant of claim 43 wherein the compressible medium is polycarbonate urethane.

105. The implant of claim 49 wherein the compressible medium is polycarbonate urethane.

106. The implant of claim 60 wherein the compressible medium is polycarbonate urethane.

107. The implant of claim 85 wherein the compressible medium is polycarbonate urethane.
